(19) United States
(12) Patent Application Publication
(10) Pub. No.: US 2006/0206005 A1
(43) Pub. Date: Sep. 14, 2006

(54) Placement multi-band bioassay device

(76) Inventors: Mang Ou-Yang, Hsinchu City (TW); Shih-Chieh Lu, Hsinchu City (TW); Hsien-Ming Wu, Hsinchu City (TW); Ker-Jer Huang, Hsinchu City (TW); Ping-Kuo Weng, Hsinchu City (TW); Tah-Yeong Lin, Hsinchu City (TW); Yung-Lin Chen, Hsinchu City (TW); Hsin-Hung Lee, Hsinchu City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/252,555

(22) Filed: Oct. 19, 2005

(30) Foreign Application Priority Data
Mar. 11, 2005 (TW) 094107446

(51) Int. Cl. A61B 1/06; A61B 5/00

(52) U.S. Cl. 600/160; 600/310

(57) ABSTRACT

A placement multi-band bioassay device makes use of a light source illuminating by several specific wavelengths, or several tunable wavelengths, or employs a device capable of capturing spectral medical images of specific bands inside the human body, e.g., the digestive system, the oral cavity, the nasal cavity, the anus, and the vagina, so as to decide the nidus. Higher discrimination rates can be obtained for some diseases or supernumerary cells to accomplish the object of “prevention is better than cure”.

### BACKGROUND OF THE INVENTION

1. Field of the Invention

The present invention relates to a multi-band bioassay device and, particularly, to a device making use of spectral images of specific bands for bioassay.

2. Description of Related Art

A conventional way of diagnosing gastrointestinal diseases makes use of an endoscope for examining gastrointestinal tissues. A viewing device with a long soft tube is inserted directly from the oral cavity to the observed gastrointestinal tissue of a patient. The front end of the soft tube with a small camera module taken image data in the gastrointestinal tissues transmits medical images to an external viewing equipment. When inserting the long soft tube, however, it is necessary to make an extra local anesthesia to reduce the discomfort of the patient. Moreover, the tube needs to be cleaned before and after each time of the examination to avoid mutual infection, hence resulting in cumbersome preparation work.

In order to improve the above drawbacks in the prior art, a capsule endoscope 2 has been proposed. As shown in FIG. 2, the capsule endoscope 2 comprises a capsule shell 4, an image acquisition module 6, an image transmission module 8, and a precision battery module 10. The image acquisition module 6, the image transmission module 8 and the precision battery module 10 are received in the capsule shell 4. The image acquisition module 6 has an LED light source 12 and a CMOS component 14. When examining the gastrointestinal tissues of a patient, a receiving device (not shown) is first worn on the patient. After the patient swallows the capsule endoscope 2, the image of the gastrointestinal tissues is illuminated by light generated by the LED light source 12 and then reflected onto the CMOS component 14. Next, the image transmission module 8 transmits the image of the gastrointestinal tissues acquired by the image acquisition module 6 to an external receiving device. After relevant examinations of the gastrointestinal tissues is performed, the capsule endoscope 2 passes the gastrointestinal system and is finally excreted along with shit.

The above conventional capsule endoscope uses a white LED as the LED light source 12 (i.e., using a light source with a continuous spectrum of visible light or mixing two or more monochromatic light sources). The CMOS 14 is used in the capsule endoscope system to acquire image data of the gastrointestinal tissues of the human body for medical examination. The prior-art endoscope, however, has only a single white LED light source for illumination and a single CMOS image sensor, but has no band selector. Therefore, the system can only acquire the full-spectrum images of the internal tissues inside the human body. Generally, these images must be interpreted by well-training medical professionals. Nonetheless, pathological changes, especially early-phase pathological changes of cancer cells, cannot be discriminated effectively.

Accordingly, the present invention proposes a placement multi-band bioassay device to solve the above problems in the prior art.

### SUMMARY OF THE INVENTION

An object of the present invention is to provide a placement multi-band bioassay device, which makes use of an illumination light source generated by several specific wavelengths or several tunable wavelengths, or employs a device capable of capturing spectral medical images of specific bands inside the human body, e.g., the digestive system, the oral cavity, the nasal cavity, the anus, and the vagina, so as to decide the nidus.

Another object of the present invention is to provide a placemen multi-band bioassay device, which enhances the discrimination ratio of pathological cells by means of timing control of different bands of the light source and different spectral responses to pathological tissues, thereby improving the disadvantage that the conventional device can only observe the full-spectrum images.

To achieve the above objects, the present invention provides a placement multi-band bioassay device, which comprises a ring image acquisition device, a specific-band beam splitter, and an image sensor. The ring image acquisition device acquires a light source reflected by a measured object to form a ring image and expanding the ring image to a ribbon image by means of optical processing. The specific-band beam splitter splits the ribbon image into several different band spectra and separately processing and then transmitting out the different band spectra. The image sensor receives the separately processed different band spectra and performs in-band series connection to the different band spectra to get a continuous image.

### BRIEF DESCRIPTION OF THE DRAWINGS

The various objects and advantages of the present invention will be more readily understood from the following detailed description when read in conjunction with the appended drawing, in which:

FIG. 1 is a perspective structure diagram of a conventional capsule endoscope;

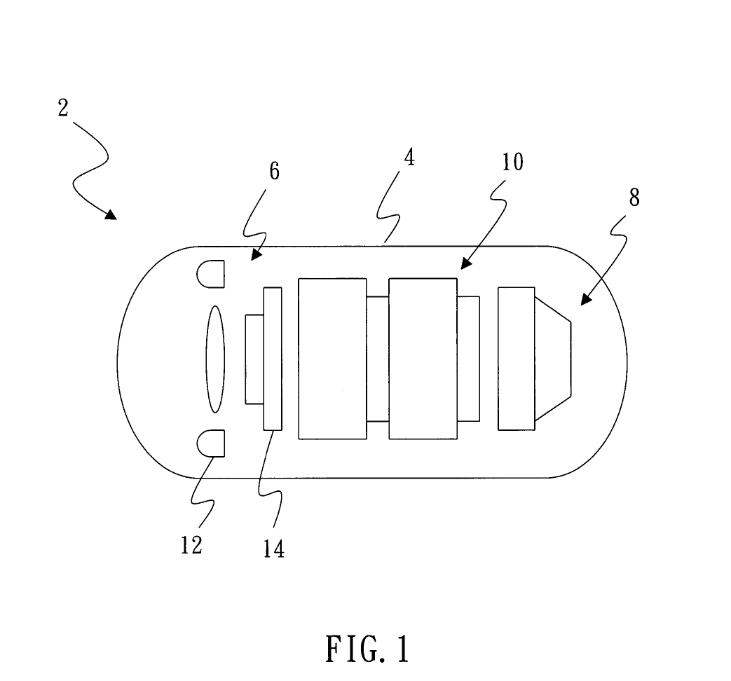

FIG. 2 is a diagram according to a first embodiment of the present invention;

FIG. 3 is a diagram according to a second embodiment of the present invention;

FIG. 4 is a diagram according to a third embodiment of the present invention; and

FIG. 5 is a diagram according to a fourth embodiment of the present invention.

### DETAILED DESCRIPTION OF THE PREFERRED EMBODIMENTS

In the past, pathological changes of tissues in the human body are examined by using endoscopes, which consists of an illumination light source is an optical lens, and an image sensor. These devices penetrating deeply into the human body transmit images of tissues to the exterior so that medical personnel can interpret these images based on their training and experiences. The endoscopes include conventional optical-fiber-tube type and recent capsule type. No matter what kind of design is adopted, the source is used by full-spectrum light (i.e., using a light source including a continuous spectrum of visible light or mixing two or more monochromatic light sources). This kind examination only acquire the full-spectrum images. However, some reasearches pointed out that various niduses stimulates the different responses in different wavelengths. For example a specific wavelength (e.g., UV light) is used to illuminate tissues of the human body, self fluorescent responses of early-phase cancer cells in the tissues of the human body can be triggered. This way of illumination can therefore provide higher discrimination ratios.

The present invention provides a placement multi-band bioassay device, which can be used cooperatively with conventional optical fiber tube type or capsule type endoscopes. The present invention will be exemplified below with the capsule endoscope.

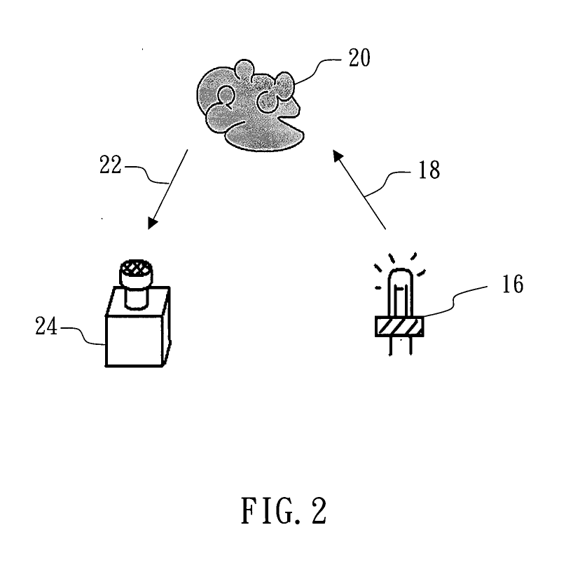

FIG. 2 is a diagram according to a first embodiment of the present invention. As shown in FIG. 2, the placement multi-band bioassay device comprises a specific light source generator 16 and an image acquisition device 24. The specific light source generator 16 can be an LED or a laser-diode. The image acquisition device 24 can be a CCD image sensor or a CMOS image sensor. The specific light source generator can make use of a filter, gradient variation of a coated film, variation of an incident angle, an optical grating, a photonic crystal, or a Fabry-Perot method to generate a specific band light source 18, and can tune the band of the light source 18 according to measuring requirement. After the specific light source generator 16 produces the specific band light source 18 and illuminates the light source 18 onto a measured object 20 in the human body, the measured object 20 will absorb light of the light source 18 to produce a band spectrum 22. The spectrum 22 is received by the image acquisition device 24 to generate an image signal. This image signal can therefore be analyzed to determine whether the tissues in the human body are normal or not.

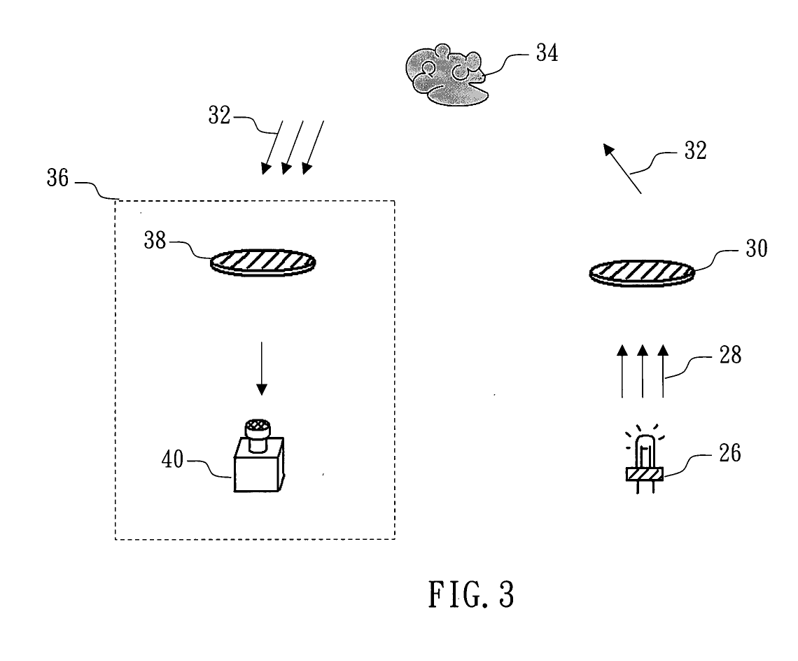

In the above first embodiment, the specific band light source 18 is directed used to illuminate the measured object 20. In the present invention, a continuous band light source can also be converted to a specific band spectrum by a band generator. FIG. 3 is a diagram according to a second embodiment of the present invention. As shown in FIG. 3, a light source generator 26 produces a continuous band light source 28. The light source generator 26 can be a gas discharge lamp, an incandescent lamp, or a common white light LED. A band generator 30 filters or converts the light source 28 to a specific band spectrum 32A, and then illuminates the specific band spectrum 32A onto a measured object 34. The measured object 34 absorbs light of this spectrum 32A and reflects a spectrum 32B to an image acquisition device 36. Based on different excitation spectra, the reflection spectrum 32B can be considered as a continuous band reflection light, and a band acquisition step can be proceeded beforehand. Therefore, the image acquisition device 36 include a band receiver 38 and an image receiver 40. The band receiver 38 receives the spectrum 32B, sifts a specific band spectrum out of the spectrum 32B, and transmits the specific band spectrum to the image receiver 40 to produce an image signal. The band receiver 38 is a device for sifting a specific band spectrum and selecting a band spectrum that enters the image receiver 40. The image receiver 40 is an image sensor such as a CMOS image sensor or a CCD image sensor.

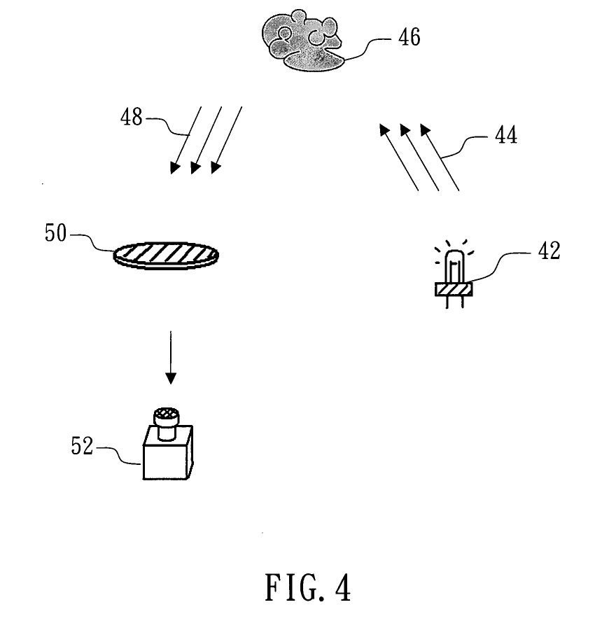

FIG. 4 is a diagram according to a third embodiment of the present invention. This embodiment is different from the above first and second embodiments in that a continuous band light source 44 directly illuminates a measured object without being converted to a specific band. A light source generator 42 is used to generate the continuous band light source 44. The measured object 46 absorbs light of the light source 44 to produce a band spectrum 48 and then reflects the spectrum 48 to a band receiver 50. The band receiver 50 can sift a specific band spectrum out of the spectrum 48 and transmit the specific band spectrum to an image acquisition device 52 to produce an image signal.

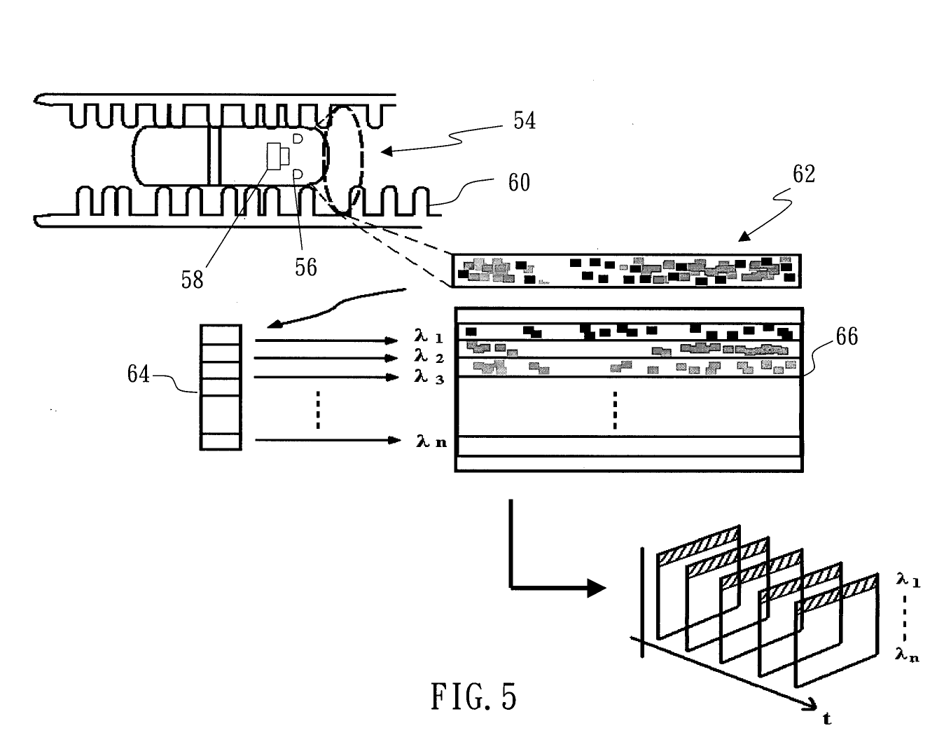

The present invention can also use a specific band or continuous band light source to acquire a ring image. FIG. 5 is a diagram according to a fourth embodiment of the present invention. As shown in FIG. 5, a placement multi-band bioassay device of the present invention comprises a ring image acquisition device 54, a specific-band beam splitter 64, and an image sensor 66. The ring image acquisition device 54 include a light source generator 56 and an image acquisition device 58. The light source generator 56 produces a continuous band light source to illuminate a measured object 60. The measured object 60 absorbs light of the light source to produce a spectrum and reflects the spectrum to the image acquisition device 58. The image acquisition device 58 processes this spectrum to produce a ring image signal and expands the ring image signal to a ribbon image 62 by means of optical processing. The specific-band beam splitter 64 splits the ribbon image 62 into several different band spectra (e.g., λ₁, λ₂, . . . , λₙ bands) and separately processes and then transmits out these different band spectra to the image sensor 66. The image sensor 66 receives these separately processed different band spectra to obtain different band images at the same time at the same place. Through image processing, the image sensor can perform in-band series connection and out-of-band separation to these different band images to get a continuous image at different bands. In other words, a continuous image signal in the measured tissues at each band can be obtained. The ring image acquisition device 54 forms the ring image by using a ring light source or a ring lens, or by blackening the center of an optical protection cover of the light source generator to allow only the ring part of the optical protection cover to be pervious to light.

To sum up, present invention proposes a placement multi-band bioassay device, which enhances the discrimination ratio of pathological cells by means of timing control of different bands of the light source and different spectral responses to pathological tissues, thereby improving the disadvantage that the conventional device can only observe common wide-band images. The present invention can widely apply to examination of the large intestine, the colon, the bronchus, and the cervix to achieved conspicuous effects.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. A placement multi-band bioassay device capable of being placed inside a human body to measure organs, said placement multi-band bioassay device comprising:
a specific light source generator capable of generating a specific band light source, said light source illuminating a measured object, said measured object absorbing light of said light source to produce and reflect an optical spectrum; and
an image acquisition device for receiving and then processing said optical spectrum to generate an image signal.

2. The placement multi-band bioassay device as claimed in claim 1, wherein said specific light source generator comprises a light source generator capable of producing a continuous-band light source and a band generator, and said band generator can filter or convert said continuous-band light source to a specific optical spectrum and then transmit and illuminate said specific optical spectrum onto said measured object.

3. The placement multi-band bioassay device as claimed in claim 2, wherein said light source generator is a gas discharge lamp or an incandescent lamp.

4. The placement multi-band bioassay device as claimed in claim 1, wherein said image acquisition device comprises an image receiver and a band receiver, and said band receiver can receive said optical spectrum, sift out a specific band optical spectrum, said then transmit said specific band optical spectrum to said image receiver.

5. The placement multi-band bioassay device as claimed in claim 4, wherein said image receiver is a CCD image sensor, a CMOS image sensor, a PIN image sensor, an APD image sensor, or other solid-state image sensors.

6. The placement multi-band bioassay device as claimed in claim 1, wherein said specific light source generator is an LED, a laser diode, or other optoelectric light source.

7. The placement multi-band bioassay device as claimed in claim 1, wherein said specific light source generator can make use of a filter, gradient variation of a coated film, variation of an incident angle, an optical grating, a photonic crystal, or a Fabry-Perot method to generate said specific band light source.

8. The placement multi-band. bioassay device as claimed in claim 1, wherein said specific light source generator can use a Fabry-Perot laser to produce a variable band light source.

9. The placement multi-band bioassay device as claimed in claim 1, wherein said image acquisition device is a CCD image sensor, a CMOS image sensor, a PIN image sensor, an APD image sensor, or other solid-state image sensors.

10. A placement multi-band bioassay device capable of being placed inside a human body to measure organs, said placement multi-band bioassay device comprising:
a light source generator capable of producing a continuous-band light source, said light source illuminating a measured object, said measured object absorbing said light source to produce and reflect an optical spectrum;
a band receiver for receiving said optical spectrum and sifting a specific band optical spectrum out of said optical spectrum; and
an image acquisition device for receiving said optical spectrum processed by said band receiver and then processing said optical spectrum to generate an image signal.

11. The placement multi-band bioassay device as claimed in claim 10, wherein said light source generator is a gas discharge lamp or an incandescent lamp.

12. The placement multi-band bioassay device as claimed in claim 10, wherein said image acquisition device is a CCD image sensor, a CMOS image sensor, a PIN image sensor, an APD image sensor, or other solid-state image sensors.

13. A placement multi-band bioassay device capable of being placed inside a human body to measure organs, said placement multi-band bioassay device comprising:
a ring image acquisition device capable of acquiring a light source reflected by a measured object to form a ring image and expanding said ring image to a ribbon image by means of optical processing;
a specific-band beam splitter capable of splitting said ribbon image into several different band spectra and separately processing and then transmitting out said different band spectra; and
an image sensor capable of receiving said separately processed different band spectra and performing in-band series connection to said different band spectra to get a continuous image.

14. The placement multi-band bioassay device as claimed in claim 13, wherein said ring image acquisition device comprises a light source generator and an image acquisition device.

15. The placement multi-band bioassay device as claimed in claim 14, wherein said light source generator can produce a specific or continuous band light source to illuminate a measured object, said measured object absorbing light of said light source to produce and reflect an optical spectrum to said image acquisition device, and said image acquisition device processes said optical spectrum to produce a ring image signal and then expands said ring image signal to a ribbon image by means of optical processing.

16. The placement multi-band bioassay device as claimed in claim 13, wherein said image acquisition device is a CCD image sensor, a CMOS image sensor, a PIN image sensor, an APD image sensor, or other solid-state image sensors.

17. The placement multi-band bioassay device as claimed in claim 13, wherein said image acquisition device forms said ring image by using a ring light source or a ring lens, or by blackening the center of an optical protection cover of said light source generator to allow only the ring part of said optical protection cover to be pervious to light.
